(19) United States
(12) Patent Application Publication
(10) Pub. No.: US 2023/0329172 A1
(43) Pub. Date: Oct. 19, 2023

(54) Sunflower Plant

(71) Applicant: TAKII & Co., Ltd., Kyoto-shi (JP)

(72) Inventors: Hideaki Ikeguchi, Kyoto-shi (JP); Takahiro Sendo, Kyoto-shi (JP); Hitomi Aoike, Kyoto-shi (JP)

(73) Assignee: TAKII & Co., Ltd, Kyoto-shi (JP)

(21) Appl. No.: 18/122,900

(22) Filed: Mar. 17, 2023

(30) Foreign Application Priority Data
Mar. 17, 2022 (JP) 2022-042597
Mar. 2, 2023 (JP) 2023-032214

(51) Int. Cl. A01H 6/14; A01H 5/02

(52) U.S. Cl. CPC A01H 6/1416; A01H 5/02

(57) ABSTRACT

The present disclosure provides a novel sunflower variety. The sunflower plant of the present disclosure is grown from a seed of a sunflower plant identified by Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, or Accession No. FERM BP-22451.

### TECHNICAL FIELD

The present disclosure relates to a sunflower plant.

### BACKGROUND ART

Sunflower plants (Helianthus annuus L.) are annual or perennial herbs that belong to the family Asteraceae and are native to southwestern North America. These species are cultivated for oil all over the world, and also, many varieties of sunflowers are commercially available for ornamental purposes.

### CITATION LIST

### Non-Patent Document

Non-Patent Document 1: Vandenbrink, Joshua P et al. “Turning heads: the biology of solar tracking in sunflower.” Plant science: an international journal of experimental plant biology vol. 224 (2014): 20-6. doi: 10.1016/j.plantsci.2014.04.006

### SUMMARY OF INVENTION

### Technical Problem

Sunflower plants have easterly oriented flower stems owing to their genetic characteristic (Non-Patent Document 1). Thus, in the fields of commercial production, when bundling up a plurality of cut flowers according to the shipping standard at the time of shipment, it is necessary to align the orientation of flowers, and this requires labor and time. Also, in the case of packing sunflower plants in boxes, it is necessary to align the orientation of flowers for the same reason.

In the field of flower arrangement, especially in situations where it is desired to use a sunflower plant with its flower facing upward, it is necessary to perform a laborious operation such as correcting the bending of the flower neck with a wire. Under these circumstances, there is a need for a sunflower plant whose flower stem is oriented upward.

In light of the foregoing, it is an object of the present disclosure to provide a novel sunflower plant.

### Solution to Problem

In order to achieve the above object, a sunflower plant of the present disclosure is grown from a seed identified by Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, or Accession No. FERM BP-22451.

A sunflower plant production method of the present disclosure includes the step of selfing the sunflower plant of the present disclosure.

Another sunflower plant production method of the present disclosure includes the step of crossing the sunflower plant of the present disclosure with another sunflower plant.

### Advantageous Effects of Invention

The present disclosure can provide a novel sunflower plant.

### BRIEF DESCRIPTION OF DRAWINGS

FIG. 1 shows schematic views illustrating examples of the attitude of the head of a sunflower plant.

FIG. 2 shows schematic views each showing the inclination angle of the flower stem of a sunflower plant.

FIG. 3 shows schematic views each showing an example of the growth stage of the flower of a sunflower plant.

FIG. 4 shows photographs comparing the inclination angles of the heads of sunflower plants of respective lines.

FIG. 5 is a chart comparing the inclination angles of the heads of flowers of sunflower plants of respective lines.

### DESCRIPTION OF EMBODIMENTS

<Definition>

The term “sunflower plant” as used in the present disclosure refers to a plant of the species Helianthus annuus in the genus Helianthus belonging to the subfamily Asteroideae of the family Asteraceae. The sunflower plant may be a hybrid with a related species or a wild species, for example. Examples of the related species include Helianthus argophyllus (Silverleaf Sunflower), Helianthus debilis (Cucumberleaf Sunflower), and Helianthus giganteus (Giant Sunflower).

The term “sunflower plant for cultivation”, “sunflower variety for cultivation”, or “sunflower for cultivation” as used herein refers to a sunflower plant that is cultivated by humans and is superior in terms of cultivation or a variety, breeding line, or cultivar thereof. The “sunflower plant for cultivation”, “sunflower variety for cultivation”, or “sunflower for cultivation” may be a pure line, a hybrid thereof, or a hybrid with a related species, such as a hybrid with another sunflower plant of Helianthus argophyllus, Helianthus debilis, or Helianthus giganteus.

The term “plant body” or “plant” as used herein refers to a plant individual representing the whole plant.

The term “a part of a plant body” or “a part of a plant” as used herein refers to a part of a plant individual.

The term “crossing” as used herein refers to crossing of two parental lines. The crossing can also be referred to as crossbreeding, for example.

The present disclosure will be described below with reference to illustrative examples. It is to be noted, however, that the present disclosure is not limited to the following examples and the like, and any changes and modifications may be made therein. In the present disclosure, descriptions regarding one aspect or embodiment can also be applied to another aspect or embodiment, and vice versa, unless otherwise stated. In the present specification, when a numerical range is delimited with the use of “to”, numerical values or values representing physical quantities indicated before and after “to” are also included in this numerical range. When a plurality of numerical values are given as examples of the upper limit and/or the lower limit of a certain numerical range in the present specification, these numerical values may be used in any combination as the upper limit and the lower limit of the numerical range. When an expression like “A and/or B” is used in the present specification, it encompasses the cases of “only A”, “only B”, and “both A and B”.

<Sunflower Plant>

In one aspect, the present disclosure provides a sunflower plant whose flower stem is oriented upward. The sunflower plant of the present disclosure includes a sunflower plant that is grown from a seed identified by Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, or Accession No. FERM BP-22451, or a progeny line thereof.

In the sunflower plant of the present disclosure, the seed identified by Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, or Accession No. FERM BP-22451 is a representative sample of the sunflower plant of the present disclosure.

The sunflower plant of the present disclosure may be a part of the sunflower plant. Examples of the part of the plant include plant cells, plant protoplasts, plant cell cultures or tissue cultures from which a plant body can be regenerated, plant calli, plant clumps, plant cells isolated from the plant or a part of the plant, leaves, pollen, embryos, cotyledons, hypocotyls, roots, tips of roots (root tips), anthers, pistils, flowers, ovaries, ovules, seeds, fruits, stems, and seedlings. The part of the plant individual may be, for example, an organ, tissue, a cell, or a propagule, and any of them may be used. Examples of the organ include petals, corollas, flowers, leaves, seeds, fruits, stems, and roots. The tissue is a part of the organ, for example. Specific examples of the part of the plant individual include microspores, flowers, flower buds, pistils, anthers, pollen, ovaries, embryos, ovules, hypocotyls, embryonic sacs, egg cells, cuttings, roots, root tips, trunks, stems, leaves, petioles, pith of leaves, cotyledons, cells, meristematic cells, protoplasts, and seeds. The pollen may be mature pollen or immature pollen. The part of the plant individual may be derived from, for example, a plant at any growth stage, and may be derived from, for example, a pre-rooting individual, a post-rooting individual, a seedling, a cutting, or a mature individual. The part of the plant individual may be one type of organ, tissue, and/or cell, or two or more types of organs, tissues, and/or cells, for example.

<Deposited Line>

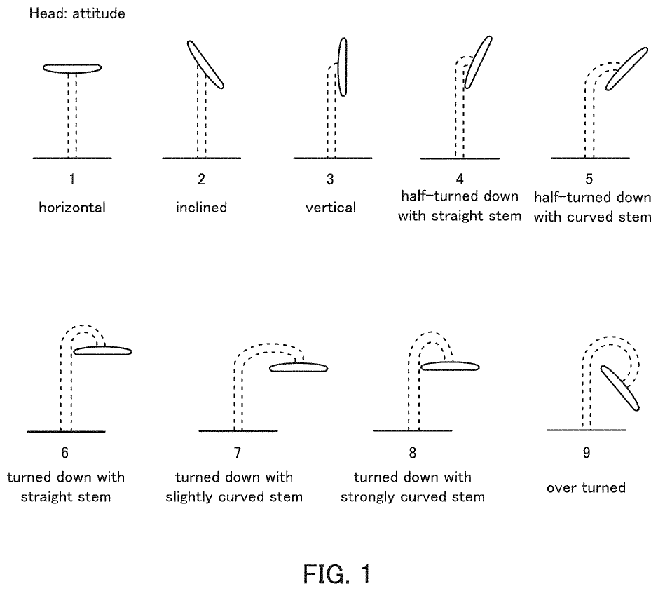

The sunflower plant of the present disclosure may be, for example, a sunflower plant (deposited line) deposited under Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, or Accession No. FERM BP-22451, or a progeny line thereof. Information on the deposit is shown below. Hereinafter, Takii 23 to Takii 30 are also referred to as Lines 1 to 8, respectively.
Type of deposit: International deposit
Name of depository institution: National Institute of Technology and Evaluation, International Patent Organism Depositary; NITE-IPOD
Address: 2-5-8-120, Kazusakamatari, Kisarazu-shi, Chiba 292-0818, Japan
(Line 1)
Accession Number: FERM BP-22444
Identifying designation: Takii 23
Date of acceptance: Feb. 10, 2022
(Line 2)
Accession Number: FERM BP-22445
Identifying designation: Takii 24
Date of acceptance: Feb. 10, 2022
(Line 3)
Accession Number: FERM BP-22446
Identifying designation: Takii 25
Date of acceptance: Feb. 10, 2022
(Line 4)
Accession Number: FERM BP-22447
Identifying designation: Takii 26
Date of acceptance: Feb. 10, 2022
(Line 5)
Accession Number: FERM BP-22448
Identifying designation: Takii 27
Date of acceptance: Feb. 10, 2022
(Line 6)
Accession Number: FERM BP-22449
Identifying designation: Takii 28
Date of acceptance: Feb. 10, 2022
(Line 7)
Accession Number: FERM BP-22450
Identifying designation: Takii 29
Date of acceptance: Feb. 10, 2022
(Line 8)
Accession Number: FERM BP-22451
Identifying designation: Takii 30
Date of acceptance: Feb. 10, 2022
The above-described deposited lines exhibit morphological and physiological characteristics shown in Table 1 below, for example. The morphological and physiological characteristics shown in Table 1 were evaluated on the basis of prototypes made in Japan in 2021. The morphological and physiological characteristics shown in Table 1 were evaluated according to the Sunflower (Helianthus L.) Test Guideline (March 2007) published by the Ministry of Agriculture, Forestry and Fisheries of Japan (MAFF). The morphological and physiological characteristics shown in Table 1 were evaluated on the basis of the evaluation criteria to be described below. Regarding the above-described morphological and physiological characteristics, reference can be made to FIG. 1.

TABLE 1

| Characteristic | | | | | | | | | |
|---|---|---|---|---|---|---|---|---|---|
| No. | Characteristics | Line 1 | Line 2 | Line 3 | Line 4 | Line 5 | Line 6 | Line 7 | Line 8 |
| 20 | Ray floret: color | 3: Medium | 3: Medium | 5: Orange | 3: Medium | 4: Orange | 3: Medium | 3: Medium | 3: Medium |
| | | yellow | yellow | | yellow | yellow | yellow | yellow | yellow |
| 21 | Disk flower: color | 1: Yellow | 1: Yellow | 3: Purple | 3: Purple | 2: Orange | 1: Yellow | 1: Yellow | 1: Yellow |
| 24 | Disk flower: | 1: Absent | 9: Present | 9: Present | 9: Present | 9: Present | 1: Absent | 9: Present | 9: Present |
| | production | | | | | | | | |
| | of pollen | | | | | | | | |
| 33 | Head: attitude | 1: Horizontal | 1: Horizontal | 1: Horizontal | 1: Horizontal | 1: Horizontal | 1: Horizontal | 1: Horizontal | 1: Horizontal |

(Characteristic No. 20)

The “Ray floret: color” refers to the color of a blooming ray floret. The “Ray floret: color” can be evaluated by visual observation of a sunflower plant at F3.2. The “Ray floret: color” can be evaluated according to the following evaluation criteria: class 1 (yellowish white), class 2 (light yellow), class 3 (medium yellow), class 4 (orange yellow), class 5 (orange), class 6 (purple), class 7 (reddish brown), and class 8 (multicolored). The sunflower at F3.2 refers to the sunflower during a period in which the three most outer rows of disk flowers have their anthers visible and detached and their stigmas unfolded.

(Characteristic No. 21)

The “Disk flower: color” refers to the color of a blooming disk flower. The “Disk flower: color” can be evaluated by visual observation of a sunflower plant at F3.2. The “Disk flower: color” can be evaluated according to the following evaluation criteria: class 1 (yellow), class 2 (orange), and class 3 (purple).

(Characteristic No. 24)

The “Disk flower: production of pollen” refers to the presence or absence of pollen production by disk flowers. The “Disk flower: production of pollen” can be evaluated by visual observation of a sunflower plant at F3.2. The “Disk flower: production of pollen” can be evaluated according to the following evaluation criteria: class 1 (absent) and class 9 (present).

(Characteristic No. 33)

The “Head: attitude” refers to the attitude of the head of a sunflower plant. The “Head: attitude” can be evaluated by visual observation of a sunflower plant at F3.2 in the case where the sunflower plant is an ornamental variety. The “Head: attitude” can be evaluated according to the following evaluation criteria, for which reference can be made to FIG. 1: class 1 (horizontal), class 2 (inclined, Ex. Var.: Taiyo), class 3 (vertical), class 4 (half-turned down with straight stem), class 5 (half-turned down with curved stem), class 6 (turned down with straight stem), class 7 (turned down with slightly curved stem), class 8 (turned down with strongly curved stem), and class 9 (over turned).

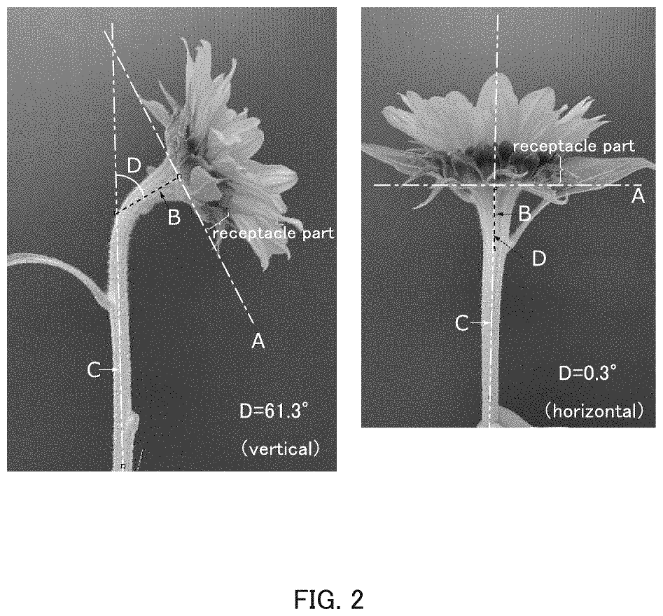

In the above-described deposited lines, flower stems are oriented upward (this characteristic is hereinafter referred to as “upward characteristic”). The orientation of a flower stem can be measured by conducting a cultivation test on a sunflower plant of interest. The cultivation test for evaluating the upward characteristic can be conducted under cultivation test conditions shown below. As shown in FIG. 2, the orientation of a flower stem of a sunflower plant of interest can be measured by measuring an angle (inclination angle of the flower stem: D) between a line (B) orthogonal to the direction (A) in which the bottom of a receptacle part of a flower extends and a line (C) indicating the axial direction of the stem. The inclination angles of flower stems can be measured under measurement conditions shown below. Then, when the median value of the inclination angles (D) of the flower stems of individuals of the sunflower plant of interest is 40° or less, 35° or less, 30° or less, 25° or less, 20° or less, 15° or less, or 10° or less, the sunflower plant of interest can be evaluated as having an upward-oriented flower stem. The above-described median value is, for example, the median value of the measured inclination angles of flower stems of 20 or more individuals of the sunflower plant of interest.

(Cultivation Test Conditions)

Cultivation start time (seeding time): April to May in general areas of Japan (a period in which a germination temperature of 20° C. to 25° C. and a growth temperature of 15° C. to 30° C. are satisfied)
Cultivation environment: unheated greenhouse
Intrarow spacing: 20 cm×20 cm
Number of rows: 4 rows
Seeding: direct seeding
Watering: From 2 weeks after germination, water is given only when wilting leaves are observed.

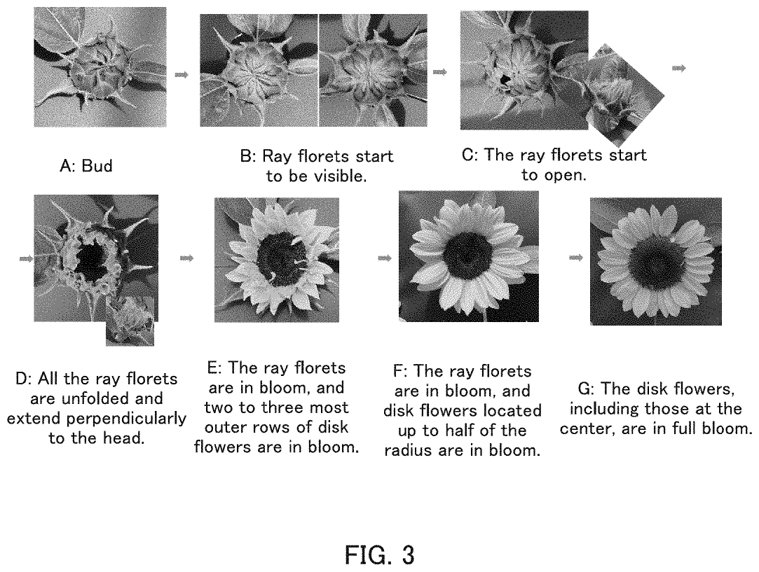

(Measurement Conditions)
Measurement period: Stages E to F shown in FIG. 3
Measurement target: Flowers located in a central portion of each ridge (individuals excluding those present in three rows at both ends of the ridge)

In the present disclosure, a plant having “essentially all physiological and morphological characteristics of a deposited line” refers to a plant having a main characteristic(s) of the deposited line when it is grown in the same environment. Examples of the main characteristic include the upward characteristic and the characteristics of the above-described Characteristic Nos. 20, 21, 24, and 33. The main characteristic is preferably the upward characteristic. The plant having “essentially all physiological and morphological characteristics of a deposited line” may be, for example, a plant having the same characteristic(s) as the deposited line, except for 5 or less, 4 or less, 3 or less, 2 or less, or 1 characteristic. In other words, the plant may have 5 or less, 4 or less, 3 or less, 2 or less, or 1 different characteristic from the deposited line. The “different characteristic from the deposited line” may be a main characteristic of the deposited line or a characteristic other than the main characteristics of the deposited line, and is preferably a characteristic other than the main characteristics of the deposited line. The “different characteristic from the deposited line” can be conferred by, for example, introduction of a characteristic and/or introduction of a gene, which will be described below. In the plant having “essentially all physiological and morphological characteristics of a deposited line”, the upward characteristic and all the characteristics of Characteristic Nos. 20, 21, 24, and 33 may be the same as those of the deposited line.

The deposited lines have, for example, single nucleotide polymorphisms (SNPs) shown in Table 2 below. In Table 2, bases in brackets [N₁/N₂] each denote a single nucleotide polymorphism. N₁ is a base (REF=F) shown in the dataset registered as Ha412HO v2.0 (the genome base sequence of the sunflower line: HA412HO)
(https://sunflowergenome.org/assembly-data/) in the sunflower genome database, and N₂ is a base (ALT=V) other than the base shown in the dataset. Regarding the analysis of the data set, reference can be made to Reference Document 1 below, for example.
Reference Document 1: Badouin H. et al., “The sunflower genome provides insights into oil metabolism, flowering and Asterid evolution” Nature, 2017, 546 (7656): 148-152.

TABLE 2

| | | | | | SEQ | | | | | | | | |
|---|---|---|---|---|---|---|---|---|---|---|---|---|---|
| | | | | SNP sequence | ID | | | | | | | | |
| SNP | chr_ | | | and its flanking | NO. | Takii | Takii | Takii | Takii | Takii | Takii | Takii | Takii |
| ID | position | REF = F | ALT = V | sequences (5′→3′) | T | 23 | 24 | 25 | 26 | 27 | 28 | 29 | 30 |
| SNP1 | Ha_chr05_ | G | A | TTGGGATTTTGGTGGTCGT | 1 | F | F | V | F | V | V | V | F |
| | 130310241 | | | CGTGGTTTTGTTGTCATGA | | | | | | | | | |
| | | | | AGGATGATGTTTTTAGGGG | | | | | | | | | |
| | | | | ATGTGGTGTTAACTCGAGG | | | | | | | | | |
| | | | | GATCTGGGTTTTGGTGGCA | | | | | | | | | |
| | | | | GTGGT[G/A]GTCCTGGTA | | | | | | | | | |
| | | | | GAGGTGACGTGAGGGGTGG | | | | | | | | | |
| | | | | CAGATTTGGTGGTCGTGAT | | | | | | | | | |
| | | | | GGCGGATTTGGAGGCCGTG | | | | | | | | | |
| | | | | GTGGAATGAGAGACGGTGG | | | | | | | | | |
| | | | | TGGGGGAAGAGGCGG | | | | | | | | | |
| SNP2 | Ha_chr11_ | T | C | ATAACATAATCTTTGTTCC | 2 | F | F | V | V | F | F | F | F |
| | 81891177 | | | AAACTGCAACGAACCCGAG | | | | | | | | | |
| | | | | ACTCGATATTAAGACGAAG | | | | | | | | | |
| | | | | ACGACAGATGACTGCACCA | | | | | | | | | |
| | | | | ACAGTGGGTATTTTGCTTA | | | | | | | | | |
| | | | | CATA[T/C]GATTTTCACG | | | | | | | | | |
| | | | | CTCCCAAGTGAACTCTAGG | | | | | | | | | |
| | | | | CTGTGACGTGCATTCCATC | | | | | | | | | |
| | | | | GAACCTTGACAAGCTTAAC | | | | | | | | | |
| | | | | ACTACTCCTCTGAGTCTTG | | | | | | | | | |
| | | | | TTAACCTTCTAATC | | | | | | | | | |
| SNP3 | Ha_chr14_ | T | A | TTTGGTAGTGTTTTACTTT | 3 | V | V | F | F | F/V | V | V | F/V/ |
| | 176065532 | | | TACCACCAAGGCCTCACCC | | | | | | | | | H |
| | | | | ACAATGGCCATACGATTAT | | | | | | | | | |
| | | | | TAGTAAAACCAAGTACATT | | | | | | | | | |
| | | | | ACAACTAGAGTTAAACCAT | | | | | | | | | |
| | | | | GTTAC[T/A]CAGTTTCTG | | | | | | | | | |
| | | | | CAACTACAAGTTTCAGTTA | | | | | | | | | |
| | | | | CAAGCCTGAATTATACATT | | | | | | | | | |
| | | | | TTTTCACGGCAAACTAACA | | | | | | | | | |
| | | | | TACTCCGATTAACCACCAA | | | | | | | | | |
| | | | | TACCCGCACCTTGCA | | | | | | | | | |
| SNP4 | Ha_chr17_ | C | A | CTGCACATGAAGTAAAAGG | 4 | F | F | V/F | V | V | F | F | V |
| | 137616549 | | | ATTAGTTCATTAAGGACTC | | | | | | | | | |
| | | | | CAAAGCCAACAGGGCTTTG | | | | | | | | | |
| | | | | GATGGAGTCTCAATAGTGT | | | | | | | | | |
| | | | | TTGACATGGATACAGTGTG | | | | | | | | | |
| | | | | ATGAA[C/A]AGTGGTTAA | | | | | | | | | |
| | | | | GTCAGGGGTTTGGACAGTT | | | | | | | | | |
| | | | | TTAGTACTGTTCCTTTCTA | | | | | | | | | |
| | | | | ACCACTATTGAGGGTCTTG | | | | | | | | | |
| | | | | CCCAATCACCTGCTGGTAA | | | | | | | | | |
| | | | | CCGAAAGGATGCCTA | | | | | | | | | |

<Progeny Line>

The sunflower plant of the present disclosure may be a progeny line of a deposited line. The progeny line may be a plant individual of the progeny line, a part of a plant individual of the progeny line, or a seed of the progeny line.

In the present disclosure, the “progeny line” or “progeny sunflower plant” (collectively referred to as “progeny line” hereinafter) is a plant obtained from a sunflower plant of a deposited line or from a progeny line thereof. In the present disclosure, the progeny line may be a plant obtained by crossing the deposited line with another deposited line or with another sunflower plant or by crossing the deposited line with a wild sunflower plant. The progeny line may be directly or indirectly obtained, obtainable, or derived from the deposited line or a progeny line thereof by selfing and/or cross-pollination, or may be derived from a parental line obtained from the deposited line using a traditional breeding method such as selfing and/or cross-pollination. The progeny line may be, for example, a selfed progeny line or a first-generation hybrid F1 (hybrid first-generation line, F1 hybrid). When obtaining the progeny line, the deposited line may be used as a female parent, a male parent, or both parents.

The crossing may be “cross-pollination” or “self-pollination”. Cross-pollination means fertilization by the union of two gametes that are derived from different plants. Self-pollination means transfer of pollen from the anthers to the stigma of the same plant. Self-pollination can also be referred to as selfing, for example. The crossing may encompass backcrossing, which is one of traditional breeding methods.

The “backcrossing” is one of traditional breeding techniques and is a method in which a breeder introduces a characteristic into a plant or a variety by repeatedly backcrossing a hybrid progeny line with one of the parental lines. A plant that includes the characteristic to be introduced may be referred to as a donor plant, for example. A plant into which the characteristic is to be introduced may be referred to as a recurrent parent, for example. The backcrossing can be performed by crossing a donor plant with a recurrent parent, whereby a first-generation hybrid F1 (hybrid first-generation line, F1 hybrid) can be obtained. Next, the progeny line having the above-described characteristic is crossed with a recurrent parent. Then, by backcrossing and/or selfing over several generations, the characteristic of the donor plant can be introduced into the recurrent parent.

In the present disclosure, the progeny line may be: regenerated from a cell culture or tissue culture, a protoplast, or a part of a plant individual, each derived from the deposited line; obtained by selfing of the deposited line; or obtained by producing seeds from a plant individual of the deposited line.

In the present disclosure, the “regeneration” refers to the development or vegetative propagation of a plant from a cell culture, a tissue culture, or a protoplast.

The “tissue culture” or “cell culture” may be a composition containing the same type or different types of isolated cells or may be a cell aggregate to be organized into a part of a plant. Tissue cultures of various tissues of sunflower plant and methods for regenerating plants from the tissue cultures are well known, and reference can be made to Reference Document 2 below, for example.
Reference Document 2: Elonore Guilley et al., “Callus formation from isolated sunflower (Helianthus annuus) mesophyll protoplasts”, Plant Cell Reports (1989) 8: 226-229

The progeny line may have desired characteristics. The progeny line may have “essentially all physiological and morphological characteristics of the deposited line” when it is cultivated under the same cultivation conditions, for example. Specifically, the progeny line may have a characteristic(s) in common with the deposited line. As specific examples, 1 or more, 2 or more, 3 or more, 4 or more, 5 or more, 6 or more, 7 or more, 8 or more, 9 or more, 10 or more, 11 or more, 12 or more, or 13 or more characteristics of the progeny line are the same as those of the deposited line. The progeny line may be a plant having the main characteristic(s) of the deposited line. Examples of the main characteristic include the upward characteristic and the characteristics of the Characteristic Nos. 20, 21, 24, and 33. The progeny line may be, for example, a plant having the same characteristic(s) as the deposited line, except for 5 or less, 4 or less, 3 or less, 2 or less, or 1 characteristic. In other words, the plant may have 5 or less, 4 or less, 3 or less, 2 or less, or 1 different characteristic from the deposited line. The “different characteristic from the deposited line” may be a main characteristic of the deposited line or a characteristic other than the main characteristics of the deposited line, and is preferably a characteristic other than the main characteristics of the deposited line. The “different characteristic from the deposited line” can be conferred by, for example, introduction of a characteristic and/or introduction of a gene, which will be described below. In the progeny line, the upward characteristic and the characteristics of Characteristic Nos. 20, 21, 24, and 33 may be the same as those of the deposited line. The different characteristic from the deposited line may be disease resistance, for example. Each characteristic can be introduced, for example, by crossing with a known plant having a locus associated with the characteristic.

The progeny line may include cells containing at least one set of chromosomes derived from the deposited line. For example, at least 6.25%, 12.5%, 25%, 30%, 35%, 40%, 45%, 50%, 55%, 60%, 65%, 70%, 75%, 80%, 85%, 90%, 95%, 96%, 97%, 98%, or 99% of alleles of the progeny line may be derived from the deposited line. That is, the progeny line may have at least about 6.25%, 12.5%, 25%, 30%, 35%, 40%, 45%, 50%, 55%, 60%, 65%, 70%, 75%, 80%, 85%, 90%, 95%, 96%, 97%, 98%, or 99% genetic complement with the corresponding deposited line.

The “allele(s)” refers to one or more genes, all of which are associated with a trait or characteristic of a sunflower plant. In a diploid cell or organism, a pair of alleles of a given gene occupy the corresponding genetic loci on a pair of homologous chromosomes.

The genetic complement can be calculated by, for example, decoding a molecular marker or base sequence, comparing it with a molecular marker or base sequence of the corresponding one of Takii 23 to Takii 30, and calculating the concordance rate. Examples of the molecular marker include SNP (single nucleotide polymorphism) markers, amplified fragment length polymorphism (AFLP) markers, restriction fragment length polymorphism (RFLP) markers, microsatellite markers, sequence-characterized amplified region (SCAR) markers, and cleaved amplified polymorphic sequence (CAPS) markers. Methods for analyzing genomes using the above-described molecular markers are well known and widely published (e.g., Reference Document 3 below). The base sequence can be decoded by, for example, extracting a chromosome from the progeny line and sequencing the chromosome. The percentage of alleles derived from the deposited line and the percentage of genetic complement may each be estimated on the basis of the number of times of crossing, for example. In this case, the percentage can be estimated on the basis of the number of times of crossing from the deposited line. As a specific example, when the number of times of crossing from the deposited line is n, the percentage can be estimated as (1/2)ⁿ×100%, for example. Reference Document 3: Talukder Z I, Gong L, Hulke B S, Pegadaraju V, Song Q, et al. (2014) A High-Density SNP Map of Sunflower Derived from RAD-Sequencing Facilitating Fine-Mapping of the Rust Resistance Gene R12. PLoS ONE 9 (7):

Preferably, the percentage of alleles derived from the deposited line and the percentage of genetic complement are each an average value of the percentages determined with respect to a plurality of progeny lines, for example. The “plurality” refers to, for example, the number of individuals sufficient to enable statistical examination, and specifically refers to, for example, at least 200 individuals and preferably 200 to 1000 individuals.

The progeny line may have an SNP(s) derived from the deposited line. The deposited line from which the progeny line is derived can be determined by, for example, evaluating the SNPs and characteristics of the progeny line in combination. The SNPs of the deposited line are the SNPs shown in Tables 2A and 2B. At least 6.25%, 12.5%, 25%, 30%, 35%, 40%, 45%, 50%, 55%, 60%, 65%, 70%, 75%, 80%, 85%, 90%, 95%, 96%, 97%, 98%, or 99% of the SNPs of the progeny line may be derived from the deposited line, for example. That is, at least about 6.25%, 12.5%, 25%, 30%, 35%, 40%, 45%, 50%, 55%, 60%, 65%, 70%, 75%, 80%, 85%, 90%, 95%, 96%, 97%, 98%, or 99% of the SNPs of the progeny line may match the SNPs of the deposited line. In the present disclosure, when, for example, 50%, 55%, 60%, 65%, 70%, 80%, 85%, 90%, 95%, 96%, 97%, 98%, or 99% or more of the SNPs of a sunflower plant of interest match the SNPs of the deposited line, the sunflower plant of interest can be determined (discriminated, estimated, appraised, or assessed) as being a progeny line of the deposited line. Of the SNPs of the deposited lines, the progeny line preferably includes an SNP(s) derived from a corresponding one of the deposited lines. As a specific example, the progeny line includes, for example, at least one SNP selected from the group consisting of SNP1 to SNP3 and SNP4. The deposited line from which the progeny line is derived can be favorably determined by, for example, evaluating the SNP1 to SNP4 and the characteristic of Characteristic No. 24 in combination. The progeny line may be evaluated by, for example, evaluating the SNP1 to SNP4 and the characteristics of Characteristic Nos. 20, 21, 24, and 33 in combination. In this case, the progeny line includes, for example, at least one selected from the group consisting of the SNP1 to SNP4 and the characteristics of Characteristic Nos. 20, 21, 24, and 33.

The progeny line may have, for example, a mutation or transgene. In this case, one or more characteristics of the progeny line are modified, for example. The progeny line can be produced by, for example, introducing mutation or a transgene into the deposited line or a progeny line thereof. The mutation may be introduced artificially or naturally. The mutation may be, for example, chemical-induced mutation or radiation-induced mutation. Also, the mutation may be introduced by, for example, a molecular biological procedure or a genome-editing technique (e.g., Reference Document 4 below). The transgene can be introduced by, for example, a method using Agrobacterium tumefaciens.
Reference Document 4: Yanfei Mao et.al., “Gene editing in plants: progress and challenges”, National Science Review, 2019, vol. 6, pp. 421-437

The at least one new characteristic may be disease resistance, for example.

The above-described transgene refers to a desired gene introduced into the genome of a plant by, for example, a genetic engineering procedure or a traditional breeding method. The transgene may be derived from the same species or a different species, for example. The transgene may be a gene including a base sequence that is the same as or different from the base sequence of the species from which it is derived. In the latter case, the different base sequence can be prepared by, for example, codon optimization of the above-described same base sequence or adding a transcriptional regulator such as a promoter to the above-described same base sequence. The transgene may have translated regions and untranslated regions.

<Haploid Plant and Doubled Haploid Plant>

The sunflower plant of the present disclosure may be a haploid plant and/or doubled haploid plant that is obtained, obtainable, or induced from a deposited line. The haploid plant and/or doubled haploid plant of a deposited line may be used in a method for producing a parental line of the deposited line. In one embodiment, the present disclosure may provide a haploid plant and/or doubled haploid plant, a plant part of a haploid plant and/or doubled haploid plant, or a seed of a haploid plant and/or doubled haploid plant.

The doubled haploid plant can be produced by doubling chromosomes in a haploid plant or a cell (e.g., Reference Document 5 below). As a specific example, haploid pollen is cultured under predetermined conditions, thereby forming plantlets with In chromosomes. Next, the plantlets are treated with, for example, a chemical substance such as colchicine to double the chromosomes. As a result, the cells of the plantlets have 2n chromosomes (doubled haploids). Then, by growing the thus-treated plantlets, the doubled haploid plants and progeny lines thereof can be obtained.
Reference Document 5: M. Todorova et al., “Doubled haploid production of sunflower (Helianthus annuus L.) through irradiated pollen-induced parthenogenesis”, Euphytica 97: 249-254, 1997.

<Method for Producing Sunflower Plant>

In another aspect, the present disclosure provides a method for producing a sunflower plant that exhibits an upward characteristic. As described above, the method for producing a sunflower plant according to the present disclosure includes the step of crossing a first sunflower plant with a second sunflower plant, and the first sunflower plant is the sunflower plant of the present disclosure.

The sunflower plant production method of the present disclosure includes the step of selfing (self-pollinating) the sunflower plant of the present disclosure. The production method of the present disclosure is characterized in that the sunflower plant of the present disclosure is selfed, and there is no particular limitation on other steps and conditions.

According to the production method of the present disclosure, a progeny line of a deposited line can be produced. Regarding the production method of the present disclosure, reference can be made to the description on the sunflower plant of the present disclosure.

In the present disclosure, the first parental line is the sunflower plant of the present disclosure, which is, for example, a sunflower plant that is grown from a seed deposited under the above-described Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, or Accession No. FERM BP-22451, or a progeny line thereof.

There is no particular limitation on the second parental line, and any sunflower plant can be used. The second parental line may be, for example, a sunflower plant of a species that is taxonomically the same as or different from the first parental line. The second parental line may be, for example, any of the above-described deposited lines or progeny line or any other sunflower plant.

The production method of the present disclosure may further include, after the crossing step, the step of growing a progeny line obtained in the crossing step, for example. Conditions for growing the progeny line in the growing step may be, for example, conditions commonly used for growing sunflower plants.

The sunflower plant of the present disclosure can be obtained by the production method of the present disclosure, for example.

<Method for Producing Seed of Sunflower Plant>

In still another aspect, the present disclosure provides a method for producing a sunflower seed. The method for producing a sunflower seed according to the present disclosure includes the steps of: selfing a sunflower plant of a deposited line or crossing the sunflower plant of the deposited line with another sunflower plant; and optionally collecting (gathering or harvesting) the resulting seeds. The seed production method of the present disclosure may provide a plant, a plant part, or a seed by growing a seed of a sunflower plant.

The seed production method of the present disclosure may be a method for producing a seed derived from a deposited line. In this case, the seed production method of the present disclosure may include the step of: (a) crossing a plant of a deposited line with another sunflower plant, thereby producing a seed. The seed production method of the present disclosure may further include the steps of: (b) cultivating a sunflower plant from the seed obtained in the step (a), thereby producing a sunflower plant derived from the deposited line; and (c) selfing the sunflower plant obtained in the step (b) or crossing the sunflower plant obtained in the step (b) with another sunflower plant, thereby producing an additional sunflower plant derived from the deposited line. The seed production method of the present disclosure may further include the step of: (d) optionally repeating the steps (b) and (c) one or more times to further produce a sunflower plant(s) derived from the deposited line. In this case, a sunflower plant to be used in a repeated step (b) as the sunflower plant cultivated from the seed obtained in the step (a) may be an additional sunflower plant obtained in the preceding step (c). The “one or more times” refer to, for example, one to ten times, three to seven times, or three to five times. The seed production method of the present disclosure may further include the step of gathering or harvesting seeds. The seed production method of the present disclosure may provide a seed produced by the above-described method and a plant or a part of a plant individual, each obtained by growing the seed.

<Method for Producing Hybrid Sunflower Plant>

In still another aspect, the present disclosure provides a method for producing a hybrid sunflower plant. The hybrid plant production method of the present disclosure includes the step of crossing the sunflower plant of the present disclosure with another sunflower plant. The hybrid plant production method of the present disclosure may further include the step of gathering or harvesting seeds obtained by crossing. The hybrid plant production method of the present disclosure may provide a seed and a hybrid plant or a part of a hybrid plant individual, each produced by the above-described method.

<Method for Introducing New Characteristic>

In still another aspect, the present disclosure provides a method for introducing at least one new trait or characteristic (collectively referred to as “characteristic” hereinafter) into a deposited line. The characteristic introduction method of the present disclosure can also be referred to as, for example, a method for producing a sunflower plant into which a new characteristic has been introduced. The characteristic introduction method of the present disclosure includes, for example, the steps of: (a) crossbreeding a plant of a deposited line with a sunflower plant including at least one new characteristic in order to produce one or more progeny lines; and (b) selecting a progeny line including the at least one new characteristic. The characteristic introduction method of the present disclosure includes, for example, the steps of: (c) crossing the progeny line with the deposited line, thereby producing a seed of a backcross progeny; and (d) selecting a backcross progeny including at least one new characteristic and essentially all physiological and morphological characteristics of the deposited line. In the steps (b) and (d), selection of the progeny line having a new characteristic may be performed by, for example, detecting the characteristic or detecting a gene or a molecular marker associated (linked) with the characteristic. The new characteristic may be disease resistance, for example.

The characteristic introduction method of the present disclosure may further include the step of: (e) optionally repeating the steps (c) and (d) one or more times to produce a sunflower plant(s) including the at least one new characteristic. In this case, in the characteristic introduction method of the present disclosure, a progeny line to be used in a repeated step (c) may be a backcross progeny selected in the preceding step (d). A sunflower plant obtained or obtainable in the step (e) may have essentially all physiological and morphological characteristics of the deposited line. Regarding the “essentially all physiological and morphological characteristics”, reference can be made to the above description on the progeny line, in which the term “progeny line” should be considered to be replaced with the term “sunflower plant obtained or obtainable in the step (e)”. The “one or more times” refer to, for example, one to ten times, three to seven times, or three to five times. The characteristic introduction method of the present disclosure may include the step of gathering or harvesting seeds. The characteristic introduction method of the present disclosure may provide a seed produced by the above-described method and a plant or a part of a plant individual, each obtained by growing the seed.

<Method for Introducing Transgene>

The present disclosure provides a method for producing a plant that is derived from a deposited line and includes at least one new trait or characteristic. The transgene introduction method of the present disclosure can also be referred to as, for example, a method for producing a sunflower plant into which a new characteristic has been introduced.

The transgene introduction method of the present disclosure includes, for example, the step of introducing mutation or a transgene that confers at least one new characteristic into a plant of a deposited line. The introduction of mutation or a transgene can be performed, for example, in the same manner as the above-described introduction of mutation or a transgene in a progeny line. The sunflower plant obtained or obtainable in the above-described introducing step may have essentially all physiological and morphological characteristics of the deposited line. Regarding the “essentially all physiological and morphological characteristics”, reference can be made to the above description on the progeny line, in which the term “progeny line” should be considered to be replaced with the term “sunflower plant obtained or obtainable in the above-described introducing step”. The transgene introduction method of the present disclosure may include the step of gathering or harvesting seeds. The transgene introduction method of the present disclosure may provide a seed produced by the above-described method and a plant or a part of a plant individual, each obtained by growing the seed. The new characteristic may be disease resistance, for example.

<Sunflower Plant Regenerated Product and Regeneration Method>

The present disclosure provides a sunflower plant regenerated from a cell culture, a tissue culture, or a protoplast of a deposited line (the regenerated sunflower plant is referred to as “regenerated product” hereinafter). The present disclosure may provide a cell culture or tissue culture of regenerable cells, or a protoplast derived from a sunflower plant of a deposited line. The cell culture, tissue culture, or protoplast may be derived from tissue including a leaf, pollen, an embryo, a cotyledon, a hypocotyl, meristematic cells, a root, a root tip, an anther, a flower, a seed, or a trunk.

The present disclosure provides a method of growth or propagation of a sunflower plant of a deposited line. The propagation of the sunflower plant of the deposited line may be vegetative propagation of the sunflower plant of the deposited line. In this case, a sunflower plant regeneration method according to the present disclosure includes, for example, the steps of: (a) collecting propagatable tissue from a plant of a deposited line; (b) culturing the tissue to obtain a grown shoot; and (c) rooting the grown shoot to obtain a rooted plantlet. The sunflower plant regeneration method of the present disclosure may further include the step of: (d) optionally growing a plant from the rooted plantlet. Regarding a method for effecting the above-described vegetative propagation, reference can be made to Reference Document 6 below, for example. The regeneration method of the present disclosure may provide, for example, a plantlet, a plant, or a part of a plant individual, each regenerated (produced) by the above-described method. The plant may have essentially all physiological and morphological characteristics of the deposited line. Regarding the “essentially all physiological and morphological characteristics”, reference can be made to the above description on the progeny line, in which the term “progeny line” should be considered to be replaced with the term “regenerated plant”.
Reference Document 6: Ibrahim Ilker Ozyigit et al., “Genotype dependent callus induction and shoot regeneration in sunflower (Helianthus annuus L.)”, African Journal of Biotechnology Vol. 6 (13), pp. 1498-1502, 4 Jul., 2007

<Harvest and Processed Product of Sunflower Plant>

In still another aspect, the present disclosure provides a harvest and/or processed product of a deposited line or a progeny line. The harvest is a whole plant or a part of a plant individual, and preferably includes: a flower; a flower and a stem (flower stem), or a seed.

When the harvest is a flower, the harvest may include a stem immediately below the flower, in addition to the flower. The length of the stem to be harvested is, for example, about 40 to 100 cm or about 60 to 80 cm. When the harvest is a flower stem, the flower stem may be a bundle of a plurality of flower stems.

The processed product encompasses any product obtained by treating a deposited line or a progeny line. The treatment is not limited to particular treatments, and examples thereof include cutting, slicing, grinding, pureeing, drying, canning, bottling, washing, packaging, freezing, and/or heating. In the deposited line or the progeny line, a plant or a part of a plant individual used in the processed product is, for example, a flower, a flower and a stem, or a seed. The processed product may be, for example, a product obtained by washing and packaging the deposited line or the progeny line. The processed product may be contained, for example, in a container of any size or shape. Specific examples of the container include bags, boxes, and cartons.

The present disclosure may provide a container containing one or more sunflower plants. The container contains a whole plant or a part of a plant individual, and preferably includes: a flower; or a flower and a stem.

The present disclosure may provide a method for producing a sunflower plant in the form of a cut flower (method for producing a cut flower). The cut flower production method of the present disclosure includes the step of gathering or harvesting a whole plant or a part of a plant individual of a deposited line or a progeny line, preferably a flower or a flower and stem of a deposited line or a progeny line, for example.

<Method for Determining Genotype>

The present disclosure provides a method for determining or detecting the genotype of a deposited line or a progeny line. The genotype determination method of the present disclosure includes, for example, the steps of: (a) obtaining a nucleic acid sample from a deposited line or a progeny line; and (b) detecting a genome in the nucleic acid sample. In the step (a), as a method for preparing the nucleic acid sample from the deposited line or the progeny line, commonly used methods for preparing a nucleic acid sample from tissue can be used. In the step (b), for example, a polymorphism(s) and/or an allele(s) in the genome in the nucleic acid sample is detected. Detection of the polymorphism(s) and/or allele(s) can be performed using, for example, SNP (single nucleotide polymorphism) genotyping, amplified fragment length polymorphism (AFLP) detection, restriction fragment length polymorphism (RFLP) identification for genomic DNA, sequence-characterized amplified region (SCAR) detection for genomic DNA, cleaved amplified polymorphic sequence (CAPS) detection for genomic DNA, random amplified polymorphic detection (RAPD) for genomic DNA, a polymerase chain reaction (PCR), DNA sequencing, an allele specific oligonucleotide (ASO) probe, or a DNA microarray. Detection of the polymorphism(s) and/or allele(s) may be performed by sequencing the base sequence of the genome or, as described above, with reference to the SNPs of the deposited line, for example. In the step (b), one polymorphism and/or allele or two or more polymorphisms and/or alleles in the genomic DNA may be detected. The genotype determination method of the present disclosure may include the step of storing the result of detecting the polymorphism(s) and/or allele(s) in a computer-readable medium. The present disclosure may provide a computer-readable medium produced by such a method.

The genotype determination method of the present disclosure may be applied to, for example, any sunflower plant (sunflower plant of interest) instead of the deposited line or the progeny line. In this case, the genotype determination method of the present disclosure may further include, for example, the step of determining whether the sunflower plant of interest is the progeny line on the basis of the result obtained in the step (b). The “determination” can also be referred to as, for example, discrimination, estimation, appraisal, or assessment. The determination can be made on the basis of, for example, the concordance rate between the result obtained in the step (b) and the genotype of the deposited line.

### EXAMPLES

The present disclosure will be described specifically below with reference to an example. It is to be noted, however, that the present disclosure is by no means limited to embodiments described in the following example.

### Example 1

Sunflower plants of the deposited lines were bred, and their traits and characteristics were examined.

(1) Breeding of Deposited Lines

In 2005, from various sunflower lines owned by TAKII & CO., LTD, sunflower lines having an upward characteristic (TAKII & CO., LTD.) were selected. In 2006, the selected sunflower lines having an upward characteristic were crossed with a plurality of sunflower lines (TAKII & CO., LTD.), and it was confirmed that progeny lines thereof had an upward characteristic. As the plurality of sunflower lines, sunflower lines having no upward characteristic and having flower colors different from each other were used. After 2007, the progeny lines resulting from crossing with these sunflower lines were repeatedly subjected to crossbreeding and selection. The selection was made on the basis of the upward characteristic, earliness, etc. In 2012 to 2016, the progeny lines were all regarded as fixed lines with their respective target characteristics being fixed, and the breeding of these eight lines was terminated.

In 2015 to 2021, the Production Department of the Head Office of TAKII & CO., LTD. seeded these fixed lines in order to examine whether they have no variation in their traits. As a result, it was confirmed that these fixed lines each have uniformity and stability. Then, seeds obtained by mass seed production of the respective fixed lines were deposited in NITE-IPOD under Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, and Accession No. FERM BP-22451.

(2) Characteristics of Deposited Line

The traits and characteristics of plant individuals of each deposited line were evaluated according to the Sunflower Variant Test Guideline published by the MAFF. Specifically, seeds of the respective deposited lines after at least three months had elapsed from being collected were seeded in the testing laboratory (Konan-shi in Shiga) on Apr. 28, 2021 and cultivated under rain cover in an unheated greenhouse. In the above-described cultivation, 30 g/m² of IB compound fertilizer No. 1 (ZEN-NOH 10-10-10) and 4 g/m² of trace-element fertilizer F.T.E (manganese: 19%, boron: 9%, TOMATEC CO., LTD.) were blended, and ridges that were 80 cm at the bottom were set up so as to extend in the north-south direction. The four-row cultivation was performed at intrarow spacing of 20 cm×20 cm. The seeds of the deposited lines were direct-seeded to a depth of about 2 cm, and after the seeding, the seeds were covered with soil from an agricultural field. After the seeds had been seeded and covered with the soil, they were sufficiently watered. From 2 weeks after germination, water was given only when wilting leaves were observed. In the above-described cultivation, in order to prevent sunflowers from falling over, a flower net with a grid size of 20 cm×20 cm was set so as to conform to the plant height. The flower net was set after the seeds had been seeded and covered with the soil. Disk flowers began to bloom from Jun. 20, 2021, and the attitude of heads of the respective deposited lines was evaluated at the flowering stage F3.2 (the stage F in FIG. 3). In order to eliminate the differences in moisture conditions and daylight conditions, sunflowers present in three rows at both ends of each ridge were not evaluated and only sunflowers located in a central portion of each ridge were evaluated. The evaluation was made on the basis of the evaluation criteria for Characteristic Nos. 20, 21, 24, and 33. The results thereof are shown in Table 3 below. For the attitude of the head as Characteristic No. 33, reference can be made to FIG. 1. Sunflower plants for comparison were cultivated and evaluated in the same manner as the plant individuals of the respective deposited lines.

TABLE 3

| | | | | Summer | | | | | | | | |
|---|---|---|---|---|---|---|---|---|---|---|---|---|
| Characteristic | | Method of | | Sunrich | Line | Line | Line | Line | Line | Line | Line | Line |
| No. | Item | investigation | Class | Orange 45 | 1 | 2 | 3 | 4 | 5 | 6 | 7 | 8 |
| 20 | Ray floret: | Observation | 1: Yellowish white, | 4 | 3 | 3 | 5 | 3 | 4 | 3 | 3 | 3 |
| | color | at F3.2 | 2: Light yellow, | | | | | | | | | |
| | | | 3: Medium yellow, | | | | | | | | | |
| | | | 4: Orange yellow, | | | | | | | | | |
| | | | 5: Orange, | | | | | | | | | |
| | | | 6: Purple, | | | | | | | | | |
| | | | 7: Reddish brown, | | | | | | | | | |
| | | | 8: Multicolored | | | | | | | | | |
| 21 | Disk flower: | Observation | 1: Yellow, | 3 | 1 | 1 | 3 | 3 | 2 | 1 | 1 | 1 |
| | color | at F3.2 | 2: Orange, | | | | | | | | | |
| | | | 3: Purple | | | | | | | | | |
| 24 | Disk flower: | Observation | 1: Absent, | 1 | 1 | 9 | 9 | 9 | 9 | 1 | 9 | 9 |
| | production of | at F3.2 | 9: Present | | | | | | | | | |
| | pollen | | | | | | | | | | | |
| 33 | Head: attitude | Observation | 1: Horizontal, | 3 | 1 | 1 | 1 | 1 | 1 | 1 | 1 | 1 |
| | | at F3.2 for | 2: Incline, | | | | | | | | | |
| | | ornamental | 3: Vertical, | | | | | | | | | |
| | | variety | 4: Half-turned down with | | | | | | | | | |
| | | | straight stem, | | | | | | | | | |
| | | | 5: Half-turned down with | | | | | | | | | |
| | | | curved stem, | | | | | | | | | |
| | | | 6: Turned down with | | | | | | | | | |
| | | | straight stem, | | | | | | | | | |
| | | | 7: Turned down with | | | | | | | | | |
| | | | slightly curved stem, | | | | | | | | | |
| | | | 8: Turned down with strongly | | | | | | | | | |
| | | | curved stem, | | | | | | | | | |
| | | | 9: Over turned | | | | | | | | | |

Summer Sunrich Orange 45: TAKII & CO., LTD.

(3) Inclination Angles of Flower Stems of Deposited Lines

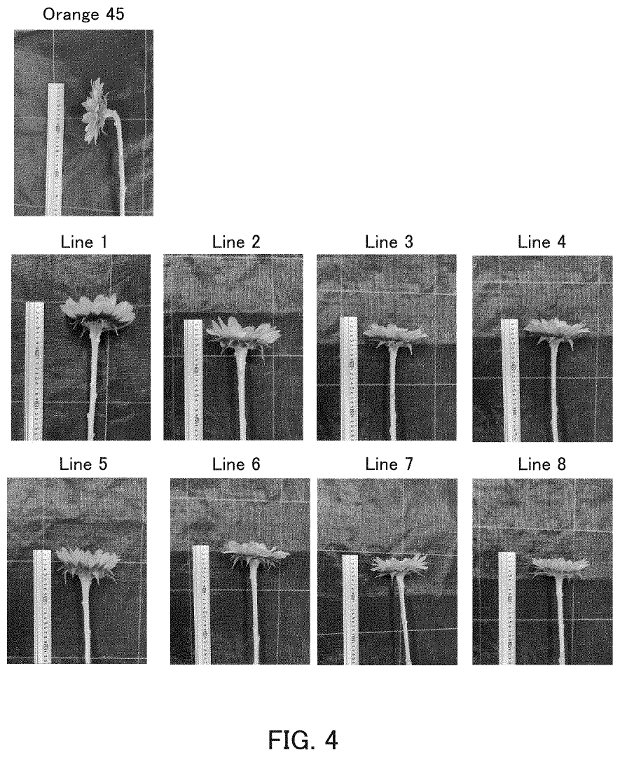

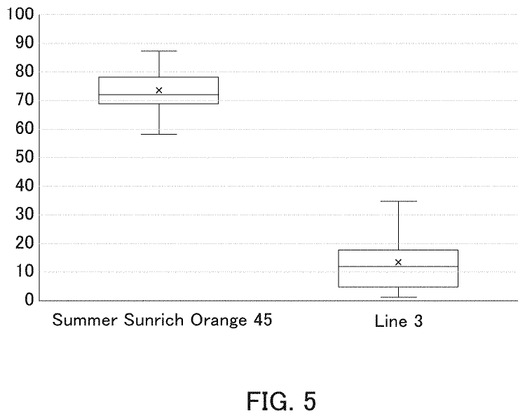

Inclination angles of flower stems of plant individuals of the respective deposited lines were examined. Specifically, regarding plant individuals (20 individuals) of each deposited line and sunflower plants for comparison (30 individuals), which were obtained by the same cultivation method as described in (2) of Example 1, inclination angles (D) of flower stems were measured. The inclination angles of the flower stems were measured in compliance with the cultivation test conditions and measurement conditions in the above-described cultivation test for the upward characteristic. The plant individuals of each deposited line and the sunflower plants for comparison in the flowering period F3.2 were photographed from the side, and the angles were measured using the Angle function of Image J. In order to eliminate the differences in moisture conditions and daylight conditions, sunflowers present in three rows at both ends of each ridge were not evaluated and the measurement was performed only for sunflowers located in a central portion of each ridge. The results thereof are shown in Tables 4 and 5 below and in FIGS. 4 and 5. Table 4 shows the measured value for a representative individual from each of the lines shown in FIG. 4.

TABLE 4

| Line name | Inclination angle (°) |
|---|---|
| Summer Sunrich Orange 45 | 80.7 |
| Line 1 | 2.5 |
| Line 2 | 0.2 |
| Line 3 | 9.0 |
| Line 4 | 3.6 |
| Line 5 | 6.5 |
| Line 6 | 5.4 |
| Line 7 | 0.2 |

Photographs shown in FIG. 4 and Table 4 compare the inclination angles of the flower stems of the sunflower plants of the respective lines. As can be seen from FIG. 4, in Lines 1 to 8, the flower stems of all the individuals exhibited an inclination angle of 40° or less and were oriented upward. On the other hand, in Summer Sunrich Orange 45 (Orange 45), the flower stem exhibited an inclination angle of more than 40° and was not oriented upward.

TABLE 5

| | Summer Sunrich Orange 45 | Line 3 |
|---|---|---|
| Average | 73.5552 | 13.4189 |
| Median value | 72.3815 | 11.9945 |
| The number of data | 30 | 20 |

FIG. 5 and Table 5 respectively show a chart and a table comparing the inclination angles of the flower stems of the sunflower plants of the respective lines. In FIG. 5, the vertical axis indicates the inclination angle of the flower stem, and the horizontal axis indicates the line of the sunflower plant. As can be seen from FIG. 5 and Table 5, the inclination angles (D) of the flower stems of Line 3 had an average value of about 13.4° and a median value of about 12.0°, and these results indicate that the flower stems of Line 3 were oriented upward as compared with the sunflower plants for comparison. Also, in the other lines, the median values were 20° or less and the flower stems were oriented upward.

(3) SNP Markers of Deposited Lines

For sunflower plants of the deposited lines and sunflower plants of commercial varieties, SNPs were designed in the entire genomic region and each SNP was analyzed. DNAs were extracted from the sunflower plants listed in Table 6 below and analyzed for SNPs using the SNP markers listed in Table 2 above. In Table 2, bases in brackets [N₁/N₂] each denote a single nucleotide polymorphism. N₁ is a base (REF=F) shown in the dataset registered as Ha412HO v2.0 (https://sunflowergenome.org/assembly-data/) in the sunflower genome database, and N₂ is a base (ALT=V) other than the base shown in the dataset. The results thereof are shown in Table 6 below. In Table 6, “F” indicates the presence of each SNP in a homozygous form of reference polymorphisms, “V” indicates the presence of each SNP in a homozygous form of polymorphisms other than N₁, and “H” indicates the presence of each SNP in a heterozygous form of a reference polymorphism and a polymorphism other than Ni. As can be seen from Table 6 below, it was found that, by checking the presence or absence of SNP1 to SNP4 and production of pollen, it is possible to distinguish the sunflower plants of the deposited lines from the sunflower plants of the commercial varieties and to identify the origins of progeny lines.
(TAKII & CO., LTD.)
Orange 45, Sunrich Orange, and Sunrich Valencia 50
(SeedSense Ltd)
ProCut White Night
(SAKATA SEED CORPORATION)
Vincent Orange, Vincent Navel, and Vincent Clear Orange

TABLE 6

| | | | | Takii | Takii | Takii | Takii | Takii | Takii | Takii |
|---|---|---|---|---|---|---|---|---|---|---|
| SNP ID | chr_position | REF = F | ALT = V | 23 | 24 | 25 | 26 | 27 | 28 | 29 |
| SNP1 | Ha_chr05_130310241 | G | A | F | F | V | F | V | V | V |
| SNP2 | Ha_chr11_81891177 | T | C | F | F | V | V | F | F | F |
| SNP3 | Ha_chr14_176065532 | T | A | V | V | F | F | F/V | V | V |
| SNP4 | Ha_chr17_137616549 | C | A | F | F | V/F | V | V | F | F |

| | | | | | | | | |
|---|---|---|---|---|---|---|---|---|
| Characteristic | Disk flower: | 1 | 9 | 9 | 9 | 9 | 1 | 9 |
| No. 24 | production of pollen | | | | | | | |

| | | | | Sunrich | | ProCut | | | Vincent |
|---|---|---|---|---|---|---|---|---|---|
| | Takii | Orange | Sunrich | Valencia | ProCut | White | Vincent | Vincent | Clear |
| SNP ID | 30 | 45 | Orange | 50 | Orange | Night | Orange | Navel | Orange |
| SNP1 | F | F | F | F | F | F | F | F | F |
| SNP2 | F | V | V | V | V | F | V | V | F |
| SNP3 | F/V/H | V | F | H | F | F | F | F | F |
| SNP4 | V | V | F | F | F | F | F | F | F |
| Characteristic | 9 | | | | | | | | |
| No. 24 | | | | | | | | | |

While the present disclosure has been described above with reference to exemplary embodiments and example, the present disclosure is by no means limited thereto. Various changes and modifications that may become apparent to those skilled in the art may be made in the configuration and specifics of the present disclosure without departing from the scope of the present disclosure.

This application claims priority from Japanese Patent Application No. 2022-042597 filed on Mar. 17, 2022 and Japanese Patent Application No. 2023-032214 filed on Mar. 2, 2023. The entire disclosures of these Japanese patent applications are incorporated herein by reference.

Patents, patent applications, and references cited in the present specification are incorporated herein in their entirety by reference, as if fully and specifically set forth herein.

<Supplementary Notes>

The whole or part of the exemplary embodiments and example disclosed above can be described as, but not limited to, the following Supplementary Notes.

<Sunflower Plant>

(Supplementary Note 1)

A seed of a sunflower plant, identified by Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, or Accession No. FERM BP-22451.

(Supplementary Note 2)

A sunflower plant grown from a seed of a sunflower plant identified by Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, or Accession No. FERM BP-22451.

(Supplementary Note 3)

A sunflower plant including:
a progeny line of the sunflower plant according to Supplementary Note 2, wherein
a flower stem of the progeny line exhibits an upward characteristic.

(Supplementary Note 4)

A sunflower plant including:
a hybrid first-generation line of the sunflower plant according to Supplementary Note 2 or 3.

(Supplementary Note 5)

A part of the sunflower plant according to any one of Supplementary Notes 2 to 4.

(Supplementary Note 6)

A seed of the sunflower plant according to any one of Supplementary Notes 2 to 4.

<Method for Producing Sunflower Plant>

(Supplementary Note 7)

A method for producing a sunflower plant, including the step of:
selfing the sunflower plant according to Supplementary Note 2 or 3.

(Supplementary Note 8)

A method for producing a sunflower plant, including the step of:
crossing the sunflower plant according to Supplementary Note 2 or 3 with another sunflower plant.

(Supplementary Note 9)

The method according to Supplementary Note 7 or 8, further including the step of: collecting a seed.

<Sunflower Plant>

(Supplementary Note 10)

A seed of a sunflower variety Takii 23, Takii 24, Takii 25, Takii 26, Takii 27, Takii 28, Takii 29, or Takii 30, a representative sample of the seed being a seed of a corresponding one of sunflower plants deposited under Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, and Accession No. FERM BP-22451.

(Supplementary Note 11)

A sunflower plant of a sunflower variety Takii 23, Takii 24, Takii 25, Takii 26, Takii 27, Takii 28, Takii 29, or Takii 30, a representative sample of the sunflower plant being a seed of a corresponding one of sunflower plants deposited under Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, and Accession No. FERM BP-22451.

(Supplementary Note 12)

A sunflower plant or a part thereof, wherein
the sunflower plant or the part thereof has essentially all physiological and morphological characteristics of the corresponding sunflower plant (sunflower plant from which the sunflower plant or the part thereof is derived) according to Supplementary Note 11.

(Supplementary Note 13)

A part of the sunflower plant according to Supplementary Note 11.

(Supplementary Note 14)

A progeny sunflower plant of the sunflower plant according to Supplementary Note 11, wherein
the progeny sunflower plant includes at least 50% of alleles of the corresponding sunflower plant (sunflower plant from which the progeny sunflower plant is derived) according to Supplementary Note 11, and
the progeny sunflower plant has the following characteristic (1) or (2):
(1) when the corresponding sunflower plant according to Supplementary Note 11 is Takii 23 or Takii 28, disk flowers do not produce pollen; and
(2) when the corresponding sunflower plant according to Supplementary Note 11 is Takii 24, Takii 25, Takii 26, Takii 27, Takii 29, or Takii 30, disk flowers produce pollen.

(Supplementary Note 15)

A seed that produces the sunflower plant according to Supplementary Note 14.

(Supplementary Note 16)

A progeny sunflower plant of the sunflower plant according to Supplementary Note 11, wherein
the progeny sunflower plant includes at least 50% of alleles of the corresponding sunflower plant (sunflower plant from which the progeny sunflower plant is derived) according to Supplementary Note 11, and
in the progeny sunflower plant, at least one SNP selected from the group consisting of SNP1 to SNP3 and SNP4 is the same as that in the corresponding sunflower plant.

(Supplementary Note 17)

A seed that produces the sunflower plant according to Supplementary Note 16.

(Supplementary Note 18)

A part of the plant according to Supplementary Note 13, 14, or 16, wherein the part of the plant includes a microspore, pollen, an ovary, an ovule, an embryonic sac, an egg cell, a cutting, a root, a trunk, a leaf, a cell, or a protoplast.

(Supplementary Note 19)

A method for producing a sunflower seed, including the steps of:
selfing the sunflower plant according to Supplementary Note 11 or crossing the sunflower plant according to Supplementary Note 11 with another sunflower plant; and
obtaining (collecting) a resulting seed.

(Supplementary Note 20)

A sunflower seed derived from a sunflower plant, produced by the method according to Supplementary Note 19.

(Supplementary Note 21)

A sunflower plant or a part thereof produced by growing the sunflower seed according to Supplementary Note 20.

(Supplementary Note 22)

The sunflower plant or the part thereof according to Supplementary Note 21, wherein a representative sample of the sunflower plant or the part thereof is a seed of a sunflower plant deposited under Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, or Accession No. FERM BP-22451, and the sunflower plant or the part thereof has essentially all physiological and morphological characteristics of the corresponding sunflower variety (sunflower variety from which the sunflower plant or the part thereof is derived) Takii 23, Takii 24, Takii 25, Takii 26, Takii 27, Takii 28, Takii 29, or Takii 30.

(Supplementary Note 23)

The sunflower plant or the part thereof according to Supplementary Note 21, wherein
the sunflower plant or the part thereof includes at least 50% of alleles of a sunflower variety Takii 23, Takii 24, Takii 25, Takii 26, Takii 27, Takii 28, Takii 29, or Takii 30, a representative sample of which is a seed of a corresponding one of sunflower plants deposited under Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, and Accession No. FERM BP-22451; and
in the sunflower plant or the part thereof, at least one SNP selected from the group consisting of SNP1 to SNP3 and SNP4 is the same as that in the corresponding sunflower plant.

(Supplementary Note 24)

A method for producing a seed of a sunflower plant derived from the sunflower plant according to Supplementary Note 11, the method including the steps of:
(a) crossing a sunflower variety Takii 23, Takii 24, Takii 25, Takii 26, Takii 27, Takii 28, Takii 29, or Takii 30, a representative sample of which is a seed of a corresponding one of sunflower plants deposited under Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, and Accession No. FERM BP-22451, with another sunflower plant, thereby producing a seed;
(b) growing a sunflower plant from the seed obtained in the step (a), thereby producing a sunflower plant derived from the sunflower variety Takii 23, Takii 24, Takii 25, Takii 26, Takii 27, Takii 28, Takii 29, or Takii 30;
(c) selfing the sunflower plant obtained in the step (b) or crossing the sunflower plant obtained in the step (b) with another sunflower plant, thereby producing an additional sunflower plant derived from the sunflower variety Takii 23, Takii 24, Takii 25, Takii 26, Takii 27, Takii 28, Takii 29, or Takii 30; and
(d) optionally repeating the steps (b) and (c) one or more times to further produce a sunflower plant(s) derived from the sunflower variety Takii 23, Takii 24, Takii 25, Takii 26, Takii 27, Takii 28, Takii 29, or Takii 30, wherein a sunflower plant used in a repeated step (b) is grown from an additional sunflower plant obtained in a preceding step (c).

(Supplementary Note 25)

A seed produced by the method according to Supplementary Note 24, wherein
a flower stem of a sunflower plant grown from the seed exhibits an upward characteristic.

(Supplementary Note 26)

A sunflower plant produced by growing the seed according to Supplementary Note 25.

(Supplementary Note 27)

A seed produced by the method according to Supplementary Note 24, wherein
the seed includes at least 50% of alleles of a sunflower variety Takii 23, Takii 24, Takii 25, Takii 26, Takii 27, Takii 28, Takii 29, or Takii 30, a representative sample of which is a seed of a corresponding one of sunflower plants deposited under Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM

BP-22450, and Accession No. FERM BP-22451; and
in the sunflower plant grown from the seed, at least one SNP selected from the group consisting of SNP1 to SNP3 and SNP4 is the same as that in the corresponding sunflower plant.

(Supplementary Note 28)

A sunflower plant produced by growing the seed according to Supplementary Note 27.

(Supplementary Note 29)

A method for introducing at least one new characteristic into the sunflower plant according to Supplementary Note 11, the method including the steps of:
(a) crossing a sunflower variety Takii 23, Takii 24, Takii 25, Takii 26, Takii 27, Takii 28, Takii 29, or Takii 30, a representative sample of which is a seed of a corresponding one of sunflower plants deposited under Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, and Accession No. FERM BP-22451, with a sunflower plant having at least one new characteristic, thereby producing one or more progenies;
(b) selecting a progeny including at least one new characteristic;
(c) crossing the progeny with the sunflower variety Takii 23, Takii 24, Takii 25, Takii 26, Takii 27, Takii 28, Takii 29, or Takii 30, thereby producing one or more backcross progenies;
(d) selecting a backcross progeny including at least one new characteristic and having essentially all physiological and morphological characteristics of the corresponding sunflower variety (sunflower variety from which the backcross progeny is derived) Takii 23, Takii 24, Takii 25, Takii 26, Takii 27, Takii 28, Takii 29, or Takii 30; and
(e) optionally repeating the steps (c) and (d) one or more times to produce a sunflower plant(s) including at least one new characteristic and having essentially all physiological and morphological characteristics of the corresponding sunflower variety (sunflower variety from which the backcross progeny is derived) Takii 23, Takii 24, Takii 25, Takii 26, Takii 27, Takii 28, Takii 29, or Takii 30, wherein a sunflower plant used in a repeated step (c) is a backcross progeny selected in a preceding step (d).

(Supplementary Note 30)

A sunflower plant produced by the method according to Supplementary Note 29.

(Supplementary Note 31)

A method for introducing at least one new characteristic into the sunflower plant according to Supplementary Note 11, the method including the steps of:
(a) crossing a sunflower variety Takii 23, Takii 24, Takii 25, Takii 26, Takii 27, Takii 28, Takii 29, or Takii 30, a representative sample of which is a seed of a corresponding one of sunflower plants deposited under Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, and Accession No. FERM BP-22451, with a sunflower plant having at least one new characteristic, thereby producing one or more progenies;
(b) selecting a progeny including at least one new characteristic;
(c) crossing the progeny with the sunflower variety Takii 23, Takii 24, Takii 25, Takii 26, Takii 27, Takii 28, Takii 29, or Takii 30, thereby producing one or more backcross progenies;
(d) selecting a backcross progeny that includes at least one new characteristic and at least 50% of alleles of the corresponding sunflower variety (sunflower variety from which the backcross progeny is derived) Takii 23, Takii 24, Takii 25, Takii 26, Takii 27, Takii 28, Takii 29, or Takii 30 and in which at least one SNP selected from the group consisting of SNP1 to SNP3 and SNP4 is the same as that in the corresponding sunflower plant; and
(e) optionally repeating the steps (c) and (d) one or more times to produce a sunflower plant that includes at least one new characteristic and at least 50% of alleles of the corresponding sunflower variety (sunflower variety from which the backcross progeny is derived) Takii 23, Takii 24, Takii 25, Takii 26, Takii 27, Takii 28, Takii 29, or Takii 30 and in which at least one SNP selected from the group consisting of SNP1 to SNP3 and SNP4 is the same as that in the corresponding sunflower plant, wherein a sunflower plant used in a repeated step (c) is a backcross progeny selected in a preceding step (d).

(Supplementary Note 32)

A sunflower plant produced by the method according to Supplementary Note 31.

(Supplementary Note 33)

A method for producing a sunflower plant that is derived from a sunflower variety Takii 23, Takii 24, Takii 25, Takii 26, Takii 27, Takii 28, Takii 29, or Takii 30 and includes at least one new characteristic, the method including the step of:
introducing mutation or a transgene that confers at least one new characteristic into a sunflower variety Takii 23, Takii 24, Takii 25, Takii 26, Takii 27, Takii 28, Takii 29, or Takii 30, a representative sample of which is a seed of a corresponding one of sunflower plants deposited under Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, and Accession No. FERM BP-22451.

(Supplementary Note 34)

A sunflower plant produced by the method according to Supplementary Note 33.

(Supplementary Note 35)

A method for producing a cut flower of a sunflower plant, including the step of: harvesting a flower, or the flower and a stem, from the sunflower plant according to Supplementary Note 11 or a progeny line thereof.

(Supplementary Note 36)

A processed product of the sunflower plant of Supplementary Note 11 or a progeny line thereof, the processed product including:
a flower, the flower and a stem, or a seed, each having been cut, sliced, ground, pureed, dried, canned, bottled, washed, packaged, frozen, and/or heat-treated.

(Supplementary Note 37)

A tissue culture of regenerable cells or regenerable protoplasts derived from the sunflower plant according to Supplementary Note 11 or a progeny line thereof.

(Supplementary Note 38)

The tissue culture according to Supplementary Note 37, wherein the cells or the protoplasts are derived from a leaf, pollen, an embryo, a cotyledon, a hypocotyl, a meristematic cell, a root, a root tip, an anther, a flower, a seed, or a stem.

(Supplementary Note 39)

A sunflower plant regenerated from the tissue culture according to Supplementary Note 38.

(Supplementary Note 40)

The sunflower plant according to Supplementary Note 39, wherein
a representative sample of the sunflower plant is a seed of a sunflower plant deposited under Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, or Accession No. FERM BP-22451, and the sunflower plant has essentially all physiological and morphological characteristics of the corresponding sunflower variety (sunflower variety from which the sunflower plant is derived) Takii 23, Takii 24, Takii 25, Takii 26, Takii 27, Takii 28, Takii 29, or Takii 30.

(Supplementary Note 41)

The sunflower plant according to Supplementary Note 39, wherein
a representative sample of the sunflower plant is a seed of a sunflower plant deposited under Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, or Accession No. FERM BP-22451, the sunflower plant includes at least 50% of alleles of the corresponding sunflower variety (sunflower variety from which the backcross progeny is derived) Takii 23, Takii 24, Takii 25, Takii 26, Takii 27, Takii 28, Takii 29, or Takii 30, and in the sunflower plant, at least one SNP selected from the group consisting of SNP1 to SNP3 and SNP4 is the same as that in the corresponding sunflower plant.

(Supplementary Note 42)

A method for vegetative propagation of the sunflower plant according to Supplementary Note 11, the method including the steps of:
(a) collecting propagatable tissue from a sunflower variety Takii 23, Takii 24, Takii 25, Takii 26, Takii 27, Takii 28, Takii 29, or Takii 30, a representative sample of which is a seed of a corresponding one of sunflower plants deposited under Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, and Accession No. FERM BP-22451;
(b) culturing the tissue to obtain a grown shoot;
(c) rooting the grown shoot to obtain a rooted plantlet; and
(d) optionally growing a plant from the rooted plantlet.

(Supplementary Note 43)

A sunflower plantlet or sunflower plant produced by the method according to Supplementary Note 42.

(Supplementary Note 44)

A sunflower plantlet or sunflower plant produced by the method according to Supplementary Note 42, wherein
the sunflower plantlet or sunflower plant has essentially all physiological and morphological characteristics of the corresponding sunflower variety (sunflower variety from which the sunflower plantlet or sunflower plant is derived) Takii 23, Takii 24, Takii 25, Takii 26, Takii 27, Takii 28, Takii 29, or Takii 30, a representative sample of which is a seed of a corresponding one of sunflower plants deposited under Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, and Accession No. FERM BP-22451.

(Supplementary Note 45)

A sunflower plantlet or sunflower plant produced by the method according to

Supplementary Note 42, wherein
the sunflower plantlet or sunflower plant includes at least 50% of alleles of the corresponding sunflower variety (sunflower variety from which the sunflower plantlet or sunflower plant is derived) Takii 23, Takii 24, Takii 25, Takii 26, Takii 27, Takii 28, Takii 29, or Takii 30, a representative sample of which is a seed of a corresponding one of sunflower plants deposited under Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, and Accession No. FERM BP-22451, and in the sunflower plantlet or sunflower plant, at least one SNP selected from the group consisting of SNP1 to SNP3 and SNP4 is the same as that in the corresponding sunflower plant.

### INDUSTRIAL APPLICABILITY

As specifically described above, the present disclosure can provide a novel sunflower plant. Therefore, the present disclosure is very useful in the field of agriculture such as breeding, for example.

1. A seed of a sunflower variety Takii 23, Takii 24, Takii 25, Takii 26, Takii 27, Takii 28, Takii 29, or Takii 30, a representative sample of the seed being a seed of a corresponding one of sunflower plants deposited under Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, and Accession No. FERM BP-22451.

2. A sunflower plant of a sunflower variety Takii 23, Takii 24, Takii 25, Takii 26, Takii 27, Takii 28, Takii 29, or Takii 30, a representative sample of the sunflower plant being a seed of a corresponding one of sunflower plants deposited under Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, and Accession No. FERM BP-22451.

3. A sunflower plant or a part thereof, wherein
the sunflower plant or the part thereof has essentially all physiological and morphological characteristics of the corresponding sunflower plant (sunflower plant from which the sunflower plant or the part thereof is derived) according to claim 2.

4. A progeny sunflower plant of the sunflower plant according to claim 2, wherein
the progeny sunflower plant includes at least 50% of alleles of the corresponding sunflower plant (sunflower plant from which the progeny sunflower plant is derived) according to claim 2, and
the progeny sunflower plant has the following characteristic (1) or (2):
(1) when the corresponding sunflower plant according to claim 2 is Takii 23 or Takii 28, disk flowers do not produce pollen; and
(2) when the corresponding sunflower plant according to claim 2 is Takii 24, Takii 25, Takii 26, Takii 27, Takii 29, or Takii 30, disk flowers produce pollen.

5. A seed that produces the sunflower plant according to claim 4.

6. A progeny sunflower plant of the sunflower plant according to claim 2, wherein
the progeny sunflower plant includes at least 50% of alleles of the corresponding sunflower plant (sunflower plant from which the progeny sunflower plant is derived) according to claim 2, and
in the progeny sunflower plant, at least one SNP selected from the group consisting of SNP1 to SNP3 and SNP4 is the same as that in the corresponding sunflower plant.

7. A seed that produces the sunflower plant according to claim 6.

8. A part of the plant according to claim 6, wherein
the part of the plant includes a microspore, pollen, an ovary, an ovule, an embryonic sac, an egg cell, a cutting, a root, a trunk, a leaf, a cell, or a protoplast.

9. A method for producing a sunflower seed, comprising the steps of:
selfing the sunflower plant according to claim 2 or crossing the sunflower plant according to claim 2 with another sunflower plant; and
obtaining (collecting) a resulting seed.

10. A sunflower seed derived from a sunflower plant, produced by the method according to claim 9.

11. A sunflower plant or a part thereof produced by growing the sunflower seed according to claim 10.

12. The sunflower plant or the part thereof according to claim 11, wherein
a representative sample of the sunflower plant or the part thereof is a seed of a sunflower plant deposited under Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, or Accession No. FERM BP-22451, and the sunflower plant or the part thereof has essentially all physiological and morphological characteristics of the corresponding sunflower variety (sunflower variety from which the sunflower plant or the part thereof is derived) Takii 23, Takii 24, Takii 25, Takii 26, Takii 27, Takii 28, Takii 29, or Takii 30.

13. The sunflower plant or the part thereof according to claim 11, wherein
the sunflower plant or the part thereof includes at least 50% of alleles of a sunflower variety Takii 23, Takii 24, Takii 25, Takii 26, Takii 27, Takii 28, Takii 29, or Takii 30, a representative sample of which is a seed of a corresponding one of sunflower plants deposited under Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, and Accession No. FERM BP-22451; and
in the sunflower plant or the part thereof, at least one SNP selected from the group consisting of SNP1 to SNP3 and SNP4 is the same as that in the corresponding sunflower plant.

14. A method for introducing at least one new characteristic into the sunflower plant according to claim 2, the method comprising the steps of:
(a) crossing a sunflower variety Takii 23, Takii 24, Takii 25, Takii 26, Takii 27, Takii 28, Takii 29, or Takii 30, a representative sample of which is a seed of a corresponding one of sunflower plants deposited under Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, and Accession No. FERM BP-22451, with a sunflower plant having at least one new characteristic, thereby producing one or more progenies;
(b) selecting a progeny including at least one new characteristic;
(c) crossing the progeny with the sunflower variety Takii 23, Takii 24, Takii 25, Takii 26, Takii 27, Takii 28, Takii 29, or Takii 30, thereby producing one or more backcross progenies;
(d) selecting a backcross progeny including at least one new characteristic and having essentially all physiological and morphological characteristics of the corresponding sunflower variety (sunflower variety from which the backcross progeny is derived) Takii 23, Takii 24, Takii 25, Takii 26, Takii 27, Takii 28, Takii 29, or Takii 30; and
(e) optionally repeating the steps (c) and (d) one or more times to produce a sunflower plant(s) including at least one new characteristic and having essentially all physiological and morphological characteristics of the corresponding sunflower variety (sunflower variety from which the backcross progeny is derived) Takii 23, Takii 24, Takii 25, Takii 26, Takii 27, Takii 28, Takii 29, or Takii 30, wherein a sunflower plant used in a repeated step (c) is a backcross progeny selected in a preceding step (d).

15. A sunflower plant produced by the method according to claim 14.

16. A method for producing a sunflower plant that is derived from a sunflower variety Takii 23, Takii 24, Takii 25, Takii 26, Takii 27, Takii 28, Takii 29, or Takii 30 and includes at least one new characteristic, the method comprising the step of:
introducing mutation or a transgene that confers at least one new characteristic into a sunflower variety Takii 23, Takii 24, Takii 25, Takii 26, Takii 27, Takii 28, Takii 29, or Takii 30, a representative sample of which is a seed of a corresponding one of sunflower plants deposited under Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, and Accession No. FERM BP-22451.

17. A sunflower plant produced by the method according to claim 16.

18. A tissue culture of regenerable cells or regenerable protoplasts derived from the sunflower plant according to claim 2 or a progeny line thereof.

19. A sunflower plant regenerated from the tissue culture according to claim 18, wherein
the cells or the protoplasts are optionally derived from a leaf, pollen, an embryo, a cotyledon, a hypocotyl, a meristematic cell, a root, a root tip, an anther, a flower, a seed, or a stem.

20. A method for vegetative propagation of the sunflower plant according to claim 2, the method comprising the steps of:
(a) collecting propagatable tissue from a sunflower variety Takii 23, Takii 24, Takii 25, Takii 26, Takii 27, Takii 28, Takii 29, or Takii 30, a representative sample of which is a seed of a corresponding one of sunflower plants deposited under Accession No. FERM BP-22444, Accession No. FERM BP-22445, Accession No. FERM BP-22446, Accession No. FERM BP-22447, Accession No. FERM BP-22448, Accession No. FERM BP-22449, Accession No. FERM BP-22450, and Accession No. FERM BP-22451;
(b) culturing the tissue to obtain a grown shoot;
(c) rooting the grown shoot to obtain a rooted plantlet; and
(d) optionally growing a plant from the rooted plantlet.
